(19) United States
(12) Patent Application Publication
(10) Pub. No.: US 2012/0010565 A1
(43) Pub. Date: Jan. 12, 2012

(54) BALLOON CATHETER WITH IMPROVED TAPER SUPPORT

(75) Inventors: Stephen C. Olson, JR., Escondido, CA (US); Scott H. Perrin, Temecula, CA (US)

(73) Assignee: ABBOTT CARDIOVASCULAR SYSTEMS INC., Santa Clara, CA (US)

(21) Appl. No.: 12/833,326

(22) Filed: Jul. 9, 2010

(51) Int. Cl. A61M 25/10

(52) U.S. Cl. 604/103.05

(57) ABSTRACT

A catheter balloon having improved steerability is disclosed having a transition between a soft distal tip and a relatively stiffer working portion of a balloon carrying a stent, in the form of a support sleeve on the inner member. The support sleeve provides stiffness to the taper portion of the balloon and also allows precise location of a radio opaque marker. The support sleeve can have a varying thickness or be made of a combination of materials to yield an increasing or decreasing stiffness along the support sleeve to provide an even smoother transition along the balloon's length.

### BACKGROUND

This invention generally relates to intravascular balloon catheters such as those used in percutaneous transluminal coronary angioplasty (PTCA) and stent delivery, and more particularly to a catheter balloon with improved deliverability and more reliable positioning of radio opaque markers.

PTCA is a widely used procedure for the treatment of coronary heart disease. In this procedure, a balloon dilatation catheter is advanced into the patient's coronary artery and the balloon on the catheter is inflated within the stenotic region of the patient's artery to open up the arterial passageway and thereby increase the blood flow there through. To facilitate the advancement of the dilatation catheter into the patient's coronary artery, a guiding catheter having a preshaped distal tip is first percutaneously introduced into the cardiovascular system of a patient by the Seldinger technique or other method through the brachial or femoral arteries.

The catheter is advanced until the preshaped distal tip of the guiding catheter is disposed within the aorta adjacent the ostium of the desired coronary artery, and the distal tip of the guiding catheter is then maneuvered into the ostium. A balloon dilatation catheter may then be advanced through the guiding catheter into the patient's coronary artery over a guidewire until the balloon on the catheter is disposed within the stenotic region of the patient's artery. The balloon is inflated to open up the arterial passageway and increase the blood flow through the artery. Generally, the inflated diameter of the balloon is approximately the same diameter as the native diameter of the body lumen being dilated so as to complete the dilatation but not over expand the artery wall. After the balloon is finally deflated, blood flow resumes through the dilated artery and the dilatation catheter can be removed.

In a large number of angioplasty procedures, there may be a restenosis, i.e. reformation of the arterial plaque. To reduce the restenosis rate and to strengthen the dilated area, physicians may implant an intravascular prosthesis or “stent” inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is then deflated to remove the catheter and the stent is left in place within the artery at the site of the dilated lesion.

To accurately place the balloon at the desired location, visual markers on the balloon are utilized that are read by machines outside the body. For example, in the case where a balloon catheter is used with an fluoroscope, the radio opaque marker may be observed visually on a screen while the procedure is taking place. In many cases, the markers must be precisely located to ensure accurate placement of the balloon in the affected area. When stents are being deployed the location of the beginning and ending point of the stent can be crucial to the success of the procedure. In such cases, it is preferred that the markers be located very specifically at the junction of the body portion of the balloon with the taper portion. However, it is also important that the marker not be located on the taper portion of the balloon. Unfortunately, the manufacturing process does not readily lend itself to a precise determination as to where to apply the marker such that it is at the extreme end of the working portion of the balloon but does not extend to the taper portion.

In addition, balloon dilation and stent delivery systems are engineered to track around tortuous curves and non-linear paths of a body lumen to reach a lesion, blockage, or treatment site. Typically, in advancing the balloon catheter once the tip and distal portion of the balloon track around a curve there is a very high probability that the rest of the balloon and system will follow so as to be advanced through the vessel system. Thus, the tip of the catheter is designed to be very soft and flexible such that little force is required to torque or adjust the tip to advance the tip through a curve in the path. Conversely, the body portion of the balloon, especially when carrying a stent, is much stiffer and requires more force to push this portion of the stem around the same curve.

Between the tip and the body portion of the balloon is the taper portion. Current balloon taper portions are very flexible compared with the body portion of the balloon carrying the stent. As a result, it is not uncommon when current balloon catheters are directed through a patient's vascular that the catheter system stalls at a curve or juncture because the soft tip and distal balloon taper portion bend around a curve or juncture but the stiffer working portion carrying a stent pushes against the vessel wall defining the curve or juncture. The present invention seeks to overcome this obstacle by employing a smoother transition of stiffness along the length of the balloon between the soft tip and the stiffer stent carrying portion of the balloon.

### SUMMARY OF THE INVENTION

The present invention addresses the problem above by adding a structural support to the distal taper portion (and optionally the proximal taper section) of the catheter balloon to help the transition of the bending or flexibility between the flexible portion of the soft tip and the stiffer portion of the working section of the balloon. This structural support in the balloon taper allows a more gradual ramp in force required to transition between the soft tip and stent carrying portion of the balloon. The support in the taper portion also may be used to assist workers in the manufacturing process in aligning visual markers on the balloon's inner member with the shoulder of the balloon. The length and position of the support member is selected to precisely and repeatably align the marker at the desired location.

### BRIEF DESCRIPTION OF THE DRAWINGS

FIG. 1 is an elevated view partially in section of a balloon catheter of the present invention;

FIG. 2 is a transverse cross sectional view of the balloon catheter of FIG. 1 taken along lines 2-2;

FIG. 3 is a transverse cross sectional view of the balloon catheter of FIG. 1 taken along lines 3-3; and

FIG. 4 is an enlarged cross-sectional view of the balloon catheter of FIG. 1 with a vascular stent mounted thereon and a transitional support member of the present invention.

### DETAILED DESCRIPTION OF THE PREFERRED EMBODIMENTS

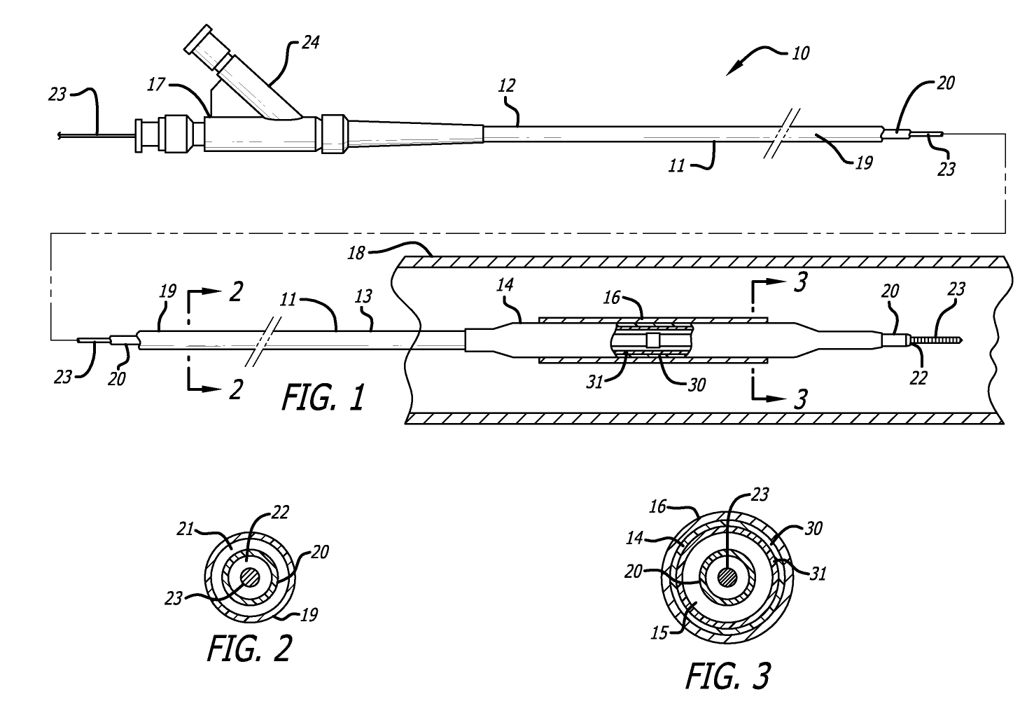

FIG. 1 shows a balloon catheter that can be used to illustrate the features of the invention. The catheter 10 of the invention generally comprises an elongated catheter shaft 11 having a proximal section 12, a distal section 13, an inflatable balloon 14 on the distal section 13 of the catheter shaft 11, and an adapter 17 mounted on the proximal section 12 of shaft 11. In FIG. 1, the catheter 10 is illustrated within a greatly enlarged view of a patient's body lumen 18, prior to expansion of the balloon 14, adjacent the tissue to be injected with therapeutic agents.

In the embodiment illustrated in FIG. 1, the catheter shaft 11 has an outer tubular member 19 and an inner tubular member 20 disposed within the outer tubular member and defining, with the outer tubular member, inflation lumen 21. Inflation lumen 21 is in fluid communication with the interior chamber 15 of the inflatable balloon 14. The inner tubular member 20 has an inner lumen 22 extending therein which is configured to slidably receive a guidewire 23 suitable for advancement through a patient's coronary arteries. The distal extremity of the inflatable balloon 14 is sealingly secured to the distal extremity of the inner tubular member 20 and the proximal extremity of the balloon is sealingly secured to the distal extremity of the outer tubular member 19.

FIGS. 2 and 3 show transverse cross sections of the catheter shaft 11 and balloon 14, respectively, illustrating the guidewire receiving lumen 22 of the guidewire's inner tubular member 20 and inflation lumen 21 leading to the balloon interior 15. The balloon 14 can be inflated by a fluid such as air, saline, or other fluid that is introduced at the port in the side arm 24 into inflation lumen 21 contained in the catheter shaft 11, or by other means, such as from a passageway formed between the outside of the catheter shaft 11 and the member forming the balloon 14, depending on the particular design of the catheter. The details and mechanics of the mode of inflating the balloon vary according to the specific design of the catheter, and are omitted from the present discussion.

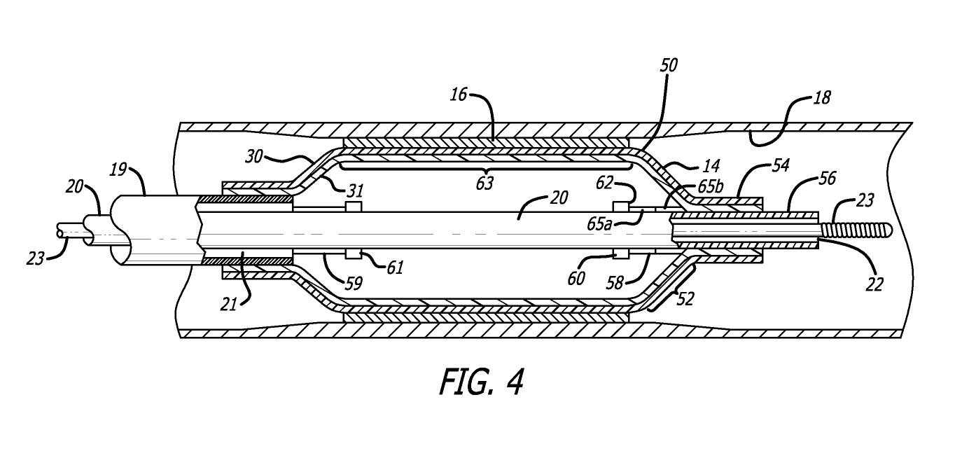

FIGS. 1 and 4 illustrate an embodiment of the catheter of FIG. 1 with a vascular stent 16 mounted thereon. The stent 16 can be made in many ways. One method of making the stent is to cut a thin-walled tubular member, such as stainless steel tubing to remove portions of the tubing in the desired pattern for the stent, leaving relatively untouched the portions of the metallic tubing which are to form the stent. The stent also can be made from other metal alloys such as tantalum, nickel-titanium, cobalt-chromium, titanium, shape memory and superelastic alloys, and the Nobel metals such as gold or platinum. It is preferred to cut the tubing in the desired pattern by means of a machine-controlled laser as is well known in the art. Stents function to hold open a segment of a blood vessel or other body lumen such as a renal or coronary artery. At present, there are numerous commercial stents being marketed throughout the world. While some of these stents are flexible and have the appropriate radial rigidity needed to hold open a vessel or artery, there typically is a tradeoff between flexibility and radial strength and the ability to tightly compress or crimp the stent onto a catheter so that it does not move relative to the catheter or dislodge prematurely prior to controlled implantation in a vessel. Currently, to secure a stent 16 on a balloon 14, after the stent is crimped onto the deflated balloon such that the balloon partially protrudes through the stent struts. During this process, the balloon and stent are placed in a heated mold and pressurized. The balloon protrusions then acts as holds to secure the stent in place.

In a typical procedure to implant stent 16, the guide wire 23 is advanced through the patient's vascular system by well known methods so that the distal end of the guide wire is advanced past the location for the placement of the stent in the body lumen 18. Prior to implanting the stent 16, the cardiologist may wish to perform an angioplasty procedure or other procedure (i.e., atherectomy) in order to open the vessel and remodel the diseased area. Thereafter, the stent delivery catheter assembly 10 is advanced over the guide wire 23 so that the stent 16 is positioned in the target area. The balloon 14 is inflated so that it expands radially outwardly and in turn expands the stent 16 radially outwardly until the stent 16 bears against the vessel wall of the body lumen 18. The balloon 14 is then deflated and the catheter withdrawn from the patient's vascular system, leaving the stent 16 in place to dilate the body lumen. The guide wire 23 typically is left in the lumen for post-dilatation procedures, if any, and subsequently is withdrawn from the patient's vascular system. As depicted in FIG. 4, the balloon 14 is fully inflated with the stent 16 expanded and pressed against the vessel wall, and thereafter the implanted stent 16 remains in the vessel after the balloon has been deflated and the catheter assembly and guide wire have been withdrawn from the patient.

FIG. 4 further illustrates a close up section of the balloon 14 showing the inner member 20 extending through the balloon's working portion 63 to the shoulder 50, taper portion 52, and out the balloon's distal end 54. The soft tip 56 is located to the distal end of the inner member 20. As can be seen, a support sleeve 58 is placed over the inner member 20 beginning at the axial location of the shoulder 50 and extending to the end of the taper portion of the balloon. The support sleeve 58 is preferably bonded to the inner member 20 and provides added stiffness to the balloon 14 through the taper portion 52. The support sleeve 58 can extend into the working portion 63 of the balloon 14 and beyond the taper portion in the distal direction. However, an advantage of the support sleeve 58 terminating at the shoulder 50 of the balloon 14 is that a radio opaque marker band 60 can be located in abutment with the support sleeve 58 and the marker band 60 will have a distal end 62 that coincides with the precise location of the shoulder 50. This allows the marker band 60 to indicate to a physician the precise location of the balloon's taper portion 52 and promote more accurate placement of the balloon's stent 16. A similar support sleeve 59 can be applied to the proximal taper portion to locate a second opaque marker band 61 such that the two markers define the working portion 63 of the balloon. In another embodiment, the marker band 60 can be placed over the support sleeve 58 in the taper portion 52 of the balloon 14 to identify the taper portion 52. A physician can then ensure that the stent is proximal to the radio opaque marker band 60 that lies in the taper section of the balloon.

The support sleeve can be made of one or more materials so as to establish either a constant or an increasing force/stiffness profile as the transition between the soft tip 56 and the stent/working body portion 63 of the balloon 14. For example, multiple rings 65a, 65b of materials increasing in stiffness can be joined together to create a multiphase transition across the sleeve 58. Alternatively, a support 59 made of a single material of varying thickness can be used to create a desired force profile. That is, the sleeve can be made thinner at the distal portion adjacent the soft tip to provide a more flexible area, while increasing in thickness in the proximal direction to ramp up to the more stiff stent/working portion 63 portion of the balloon 14.

Various materials can be used to form the support sleeve, such as materials used to make the marker band (Tungsten, Platinum/Iridium) and one or more polymers (Pebax, Nylon, etc.). The marker band 60 and support sleeve 58 can be laser bonded to each other and to the inner member 20, or heat bonding, swaging, adhesive, or other bonding methods can be used.

While particular forms of the invention have been illustrated and described, it will be apparent to those skilled in the art that various modifications can be made without departing from the spirit and scope of the invention. Accordingly, it is not intended that the invention be limited except by the appended claims.

1. A balloon catheter including a balloon attached to a catheter tubing where the balloon includes a soft distal tip, a working length, a proximal and distal taper portion, and proximal and distal shoulders that define a transition between the working length and the proximal and distal taper portions, respectively, comprising:
a support sleeve bonded to the catheter tubing having a first end aligned with the distal shoulder and a second end adjacent to the soft distal tip, the support sleeve cooperating with the distal taper portion of the balloon such that a stiffness of the support sleeve and distal taper portion is greater than a stiffness of the soft distal tip and less than a stiffness of the working length; and
a radio opaque marker abutting the support sleeve at the first end to identify the distal shoulder, wherein the support sleeve has varying stiffness along its length which translates from a lower stiffness at its second end to a higher stiffness at its first end.

2. The balloon catheter of claim 1, further comprising a second support sleeve bonded to the catheter tubing having a first end aligned with the proximal shoulder, and a radio opaque marker abutting the second support sleeve at the first end to identify the proximal shoulder.

3. The balloon catheter of claim 1, wherein the radio opaque marker is a marker band positioned about the catheter tubing.

4. The balloon catheter of claim 1, wherein the support sleeve is formed of multiple materials arranged in order to provide the transition from a higher stiffness at its first end to a lower stiffness at its second end.

5. The balloon catheter of claim 1, wherein the support sleeve is formed of a single material having a varying thickness along its length to provide the transition from a higher stiffness at its first end to a lower stiffness at its second end.

6. The balloon catheter of claim 1, wherein the support sleeve is bonded to the inner member using laser bonding.

7. The balloon catheter of claim 1, wherein the support sleeve is bonded to the inner member using heat bonding.

8. The balloon catheter of claim 1, wherein the support sleeve is bonded to the inner member using swaging.

9. The balloon catheter of claim 1, wherein the radio opaque marker is selected from Tungsten and Platinum/Iridium.

10. The balloon catheter of claim 1, wherein the support sleeve is selected from polyether block amide and nylon.

11. A balloon catheter including a balloon attached to a catheter tubing where the balloon includes a soft distal tip, a working length, a proximal and distal taper portion, and proximal and distal shoulders that define a transition between the working length and the proximal and distal taper portions, respectively, comprising:
a first support sleeve bonded to the catheter tubing having a first end aligned with the distal shoulder and a second end, the first support sleeve having a particular stiffness;
a second support sleeve bonded to the catheter tubing having a first end adjacent to the second end of the first support sleeve and a second end adjacent to the soft distal tip, the second support sleeve having a particular stiffness that is less than the stiffness of the first support sleeve, the first and second support sleeves cooperating with the distal taper portion of the balloon such that the stiffness of the second support sleeve and the distal taper portion of the balloon is greater than the stiffness of the soft distal tip and the stiffness of the first support sleeve and the distal taper portion of the balloon is less than a stiffness of the working length.

12. The balloon catheter of claim 11, wherein a radio opaque marker is located adjacent to the first end of the first support sleeve to identify the distal shoulder of the balloon.

13. The balloon catheter of claim 11, wherein the first support sleeve is formed from a different material than the second support sleeve.

14. A balloon catheter, comprising:
a catheter tubing;
a balloon attached to the catheter tubing, the balloon including a soft distal tip, a working length, a proximal and distal taper portion, and proximal and distal shoulders that define a transition between the working length and the proximal and distal taper portions; and
a support sleeve bonded to the catheter tubing having a first end aligned with the distal shoulder and a second end adjacent to the soft distal tip, the support sleeve having varying stiffness along its length which translates from a lower stiffness at its second end to a higher stiffness at its first end, the support sleeve cooperating with the distal taper portion of the balloon such that the lowest stiffness of the support sleeve and distal taper portion is greater than a stiffness of the soft distal tip and the higher stiffness of the support sleeve and distal taper portion is less than a stiffness of the working length of the balloon.

15. The balloon catheter of claim 14, wherein the support sleeve is formed of multiple materials arranged in order to provide the transition from the higher stiffness at its first end to the lower stiffness at its second end.

16. The balloon catheter of claim 14, wherein the support sleeve is formed of a single material having a varying thickness along its length to provide the transition from the higher stiffness at its first end to the lower stiffness at its second end.
